(19) United States
(12) Patent Application Publication
(10) Pub. No.: US 2025/0000676 A1
(43) Pub. Date: Jan. 2, 2025

(54) Endovascular Endoprosthesis Delivery Apparatus

(71) Applicant: Junaid QURESHI, Paradise Valley, AZ (US)

(72) Inventor: Junaid QURESHI, Paradise Valley, AZ (US)

(21) Appl. No.: 18/760,922

(22) Filed: Jul. 1, 2024

Related U.S. Application Data
(60) Provisional application No. 63/524,292, filed on Jun. 30, 2023.

(51) Int. Cl. A61F 2/95

(52) U.S. Cl. CPC A61F 2/9517; A61F 2002/9511; A61F 2250/0098

(57) ABSTRACT

The present disclosure describes an endovascular delivery apparatus that enables a radio-opaque or contrast dye to be delivered directly to the site of a clot, alleviating the need to diffuse an large amount of dye through a wound path to effectively expose a clot to a radio-opaque or contrast dye. The present invention is advantageous, as less dye may be used, thereby mitigating side-effects caused by excess dye exposure, additionally, the present invention enables dye to reach the desired location more quickly, thereby increasing the chances of patient survival during emergency stent insertion procedures.

### CLAIM OF PRIORITY

The present application includes subject matter disclosed in and claims priority to a provisional application entitled “Endovascular Endoprosthesis Delivery Apparatus” filed Jun. 30, 2023 and assigned Ser. No. 63/524,292, describing an invention made by the present inventor, herein incorporated by reference.

### BACKGROUND OF THE INVENTION

### 1. Field of the Invention

The present invention relates to an endovascular endoprosthesis delivery device, and more particularly to a device for delivering an endovascular endoprosthesis to a radiographically verifiable, desired location.

### 2. Description of Related Prior Art

A stent is typically a metal or plastic tube inserted into the lumen of an anatomic vessel or duct to keep the vessel or duct open. There are a variety of stents that serve numerous functions. For example, an expandable coronary stent is a cylindrical structure, surrounding a deflated balloon, typically used in interventional cardiology to treat narrowed or blocked coronary arteries. Expandable coronary stents are routinely threaded through blood vessels to reach an arterial blockage. Once positioned at the site of the blockage, the balloon housed within the stent is inflated, expanding the surrounding stent, thereby widening the stent's diameter, while shortening the stent's length. Due to the pressure produced by the stent's expansion, the stent pushes against the walls of the blocked artery, widening the narrowed passage. For blockages in the vascular and biliary system, expandable vascular and biliary stents function analogously to expandable coronary stents.

Traditionally, to insert a coronary stent, a catheter is initially guided through a patient's artery. A contrast dye may then be injected through the catheter, into the artery. The contrast dye may be an iodine contrast medium or other radio-opaque dye. The contrast dye enhances the visibility of blood flow, making any blockages clear and visible on a live x-ray, or angiogram. Once the blockage is identified, live X-ray images may be used to guide the stent to the sight of the blockage.

While iodine-based contrast agents are well suited for visualizing blood vessels and the cardiovascular system, such contrast agents, particularly in large quantities, may damage a patient's kidneys. Additional side effects include, but are not limited to, thyroid dysfunction, nausea, headaches, and localized irritation.

Ureteral stents function differently from expandable stents, as they are only intended for temporary use. Additionally, ureteral stents maintain their original shape and do not expand after entering the body. Ureteral stents are generally soft, hollow, silicone, or plastic tubes that function to open a blocked or narrowed ureter. The top portion of a ureteral stent sits in the kidney while the bottom end sits in the bladder, lining the entire length of the ureter to maintain an open pathway for urine to flow through.

Similarly to expandable stents, a contrast dye is often injected into a patient's urinary system to assist in ureteral stent insertion. The dye's movement through the urinary system may be observed by fluoroscopy or other such real-time medical imaging techniques. Such fluoroscopy procedures enable medical professionals to view the ureter, such that a guide wire may be properly placed within the patient's ureter. Once the guide wire is inserted, the guide wire may act as a guide for proper stent placement, as the ureteral stent may be slid over the guide wire and set within the patient's ureter.

While expandable coronary stents and ureteral stents are ubiquitous, other stents may include drug-eluting stents, bioabsorbable stents, and dual-therapy stents. As discussed above, stents are typically delivered to their target location through the assistance of a radio-opaque dye and radiographic imagery. However, such stent insertion methods are disadvantageous due to the time required for the dye to diffuse through a patient's body to the appropriate site. Often, stents are inserted during medical emergencies, such as myocardial infarctions, when time is of the essence. In such emergencies, the time delay between dye insertion, dye diffusion, and stent insertion may impact a patient's chances of successful recovery, or even survival.

Moreover, whereas a contrast dye is only medically necessary at the site of a blockage, conventional stent insertion methods often leave an excess of dye in the patient's body. The process for inserting dye for stent insertion involves injecting a radioactive dye into a wound site and allowing the dye to diffuse to the site of a blockage. Once the dye is injected, excess dye may unnecessarily diffuse throughout the body, to other tissues, systems, and organs.

Contrast dye side effects may include nausea, headache, diarrhea, rash, itchy skin, hyperthyroidism, nephrogenic systemic fibrosis, and contrast-induced nephropathy. While side effects are uncommon, and the benefits of radiographic imaging generally outweigh the risks of dye, there is a great need for the development of methods for dye insertion that minimize the amount of excess dye inserted into a patient's body, thereby mitigating and limiting contrast dye side effects. Additionally, methods for quickening the time between dye injection and stent insertion are necessary to increase patient survival during myocardial infarctions, and other medical emergencies requiring stent insertion.

### SUMMARY OF THE INVENTION

The present invention provides a stent delivery device that includes a handle section set at a proximal end of the stent delivery device, an elongated body section coupled to the handle section and set distally of the handle section, a catheter coupled to the elongated body section, and a stent coupled to the catheter such that the stent is set between the catheter and the distal end of the stent delivery device.

A plurality of channels may pass through the stent delivery device. A first channel, having two open terminals, configured to house a guide wire, passes from the handle section to the distal end of the stent delivery device. A second channel, configured to house a contrast liquid, passes from the handle section to the stent. A third channel, configured to house a deployment means, passes from the handle section to the stent deployment site.

The handle section may be configured into a multi-branched body, having a first branch that includes a first terminus for inserting a guide wire, such that the first terminus leads into the first channel. A second branch may include a second terminus, such that the second terminus may include a contrast dye injection port. The contrast dye injection port may lead into the second channel. In some embodiments, the handle section may include a third branch, such that the third branch includes a third channel, configured to house a mechanism for releasing the stent from the stent delivery device.

All three channels pass through the elongated body section. The plurality of channels passes from said handle section, into said elongated body section, and out of said elongated body section into the catheter. The plurality of channels comprises at least the first channel configured to receive a guide wire, the second channel configured to receive contrast dye, and the third channel configured to receive a stent deployment means. In some embodiments, the first channel, the second channel, and the third channel may be arranged concentrically within the elongated body section. In some embodiments, the second elongated tubular channel is the outermost channel within the elongated body section of the plurality of tubular channels. In some embodiments, the first channel, the second channel, and the third channel are arranged in parallel within the elongated body section.

The second channel may pass from the handle section, into the elongated body section, and through the catheter into the stent, whereby the stent may be inserted into a patient via a guide wire, whereby said catheter may deliver a contrast dye to an area in need of said stent.

The catheter may deliver a contrast dye to an area in need of contrast dye via at least one pore set within a side of said stent, whereby said contrast dye flows from said catheter into said stent, exiting said stent via the at least one pore, and entering the area in need of a contrast dye via the at least one pore set within the stent. In some embodiments, the stent may alternatively, or additionally be set with radio-opaque markers.

A method of inserting a stent into a bodily vessel may include inserting a guide wire set within a stent delivery apparatus into a bodily vessel; guiding the stent coupled to the stent delivery apparatus to a target area via the guide wire, injecting a contrast dye through the stent delivery apparatus, such that the contrast dye travels from a handle in the stent delivery apparatus through a channel in the stent delivery apparatus into the stent, such that the contrast dye exits the stent into the target area via at least one pore in said stent. Once the dye reaches the target area, the target area may be viewed via radiographic imaging, such that the stent may be adjusted until it is properly positioned within the target area. After the stent is properly positioned, the stent may be disengaged from the stent delivery apparatus and the guide wire and stent delivery apparatus may be removed from the target area.

### BRIEF DESCRIPTION OF THE DRAWINGS

The present invention will be described with greater specificity and clarity with reference to the following drawings, in which:

FIG. 1 illustrates a delivery catheter assembly including a delivery catheter in accordance with the invention.

FIG. 2 illustrates an enlarged detail view of region “A” of the delivery catheter as defined in FIG. 1.

FIG. 3a illustrates a cross-section view of the delivery catheter assembly of FIG. 1 taken at section line “B-B” defined in FIG. 1.

FIG. 3b illustrates a cross-section view of an alternate embodiment of a delivery catheter assembly taken at section line “B-B” defined in FIG. 1.

FIG. 3c illustrates a cross-section view of another alternate embodiment of a delivery catheter assembly taken at section line “B-B” defined in FIG. 1.

FIG. 4 illustrates a cross-section view of a handle portion of an endovascular endoprosthesis delivery device in accordance with the invention, having three ports.

### DETAILED DESCRIPTION OF THE PREFERRED EMBODIMENTS

The present invention is directed to a device and method for inserting a stent, such that, during the stent delivery procedure, a contrast dye, or radiopaque dye is quickly delivered directly to the site of a clot, thereby quickening the duration of time between dye insertion and procedure completion. Additionally, the herein described device and method directly delivers the dye to the site of a clot, thereby reducing the need to inject an excess of dye throughout a wound path. Rather, the dye is only inserted directly near the site of a clot, decreasing the side-effects association with excess dye exposure.

### General Overview

The herein described device may be referred to as stent delivery device 1, endovascular endoprosthesis delivery apparatus 1, or delivery catheter assembly 1. Starting at proximal end 38, in some embodiments, stent delivery device 1 may include a branched handle section, herein referred to as branched body 50, coupled to an elongated body section, such that the elongated body section is coupled to a catheter 4. The catheter 4 may be coupled to delivery section 17, such that stent 20 is set within delivery section 17, and such that stent 20 is set adjacent to catheter assembly 1 distal end 39. In some embodiments a plurality of channels may pass from branched body 50 through endovascular endoprosthesis delivery device 1. One such channel may house guide wire 3, such that guide wire 3 may travel through channel 30 from branched body 50 through endovascular delivery apparatus 1, ending at distal end 39. A second channel 31 may funnel contrast dye, or other such radio-opaque dyes from branched body 50 to stent 20, such that once stent 20 reaches the site of a clot, dye may travel from channel 31, through catheter 4, to delivery pores set within stent 20, such that a small amount dye may be inserted directly into the area around a clot, bypassing the majority of the wound path. A third channel 32 may function to house an endoprosthesis deployment means, such that once the stent is appropriately position, it may be disengaged from the endovascular endoprosthesis delivery device 1, such that stent delivery device 1 may be removed from a patient's body, while stent 20 remains properly positioned within the lumen of a patient's vessel, artery, organ, or other such system.

In some embodiments, a method of inserting endoprosthesis 20 into a blood vessel, may include the step of providing an endovascular endoprosthesis delivery device 1, which may include a handle section 10 at a proximal end 38 of the endovascular endoprosthesis delivery device 1. Guide wire 3 may pass through handle section 10, passing through endoprosthesis delivery device 1. In a preferred embodiment, guide wire 3 may be inserted into a patient's artery, such that stent 20 may travel over guide wire 3 until stent 20 reaches the site of an area in need of stent 20. Once stent 20 is positioned near the area of a clot, a radio-opaque contrast fluid may be injected, via endoprosthesis delivery device 1 (as further described below) into the interior of stent 20. Radiographic imaging may then be used to properly position stent 20, after which stent 20 may be disengaged from endovascular endoprosthesis delivery device 1, via a disengaging mechanism set within third channel 32. Once stent 20 is properly set and disengaged stent delivery device 1 and guide wire 3 may be removed from the patient's body. In some embodiments, stent delivery device 1 may be sterilized and set with a new guide wire, new contrast dye, and new stent and reused. In such embodiments, stent delivery device 1 may also be set with a new catheter 4. In other embodiments, stent delivery device may be disposable.

While various aspects and features of certain embodiments have been summarized above, the following detailed description illustrates a few exemplary embodiments in further detail to enable one skilled in the art to practice such embodiments. The herein-described examples are provided for illustrative purposes and are not intended to limit the scope of the invention. In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

For all figures, reference numerals and reference letters for elements described in any one figure represent the same elements as they appear and are referenced in any other figures, without requiring redundant recitation of the same description in those other figures.

A stent may include a tubular endoprosthesis device which is typically delivered and released into a bodily vessel, whereupon a tensile preload may allow the endoprosthesis to shorten in length in exchange for increasing its diameter, and residual tensile forces in the device may exert gentle but enduring radially outward forces upon the interior of the vessel so as to maintain patency in the bodily vessel over the long term. The vessel may be a blood vessel, but stents may also be deployed in bile ducts, ureters, bronchi, and any other such vessels where any such vessels are at risk of rupture, blockage, or collapse. In this specification, the word “stent” most often refers to an endoprosthesis device which is a vascular stent.

The structure and manufacture of the endoprosthesis itself may include but are not limited to, expanded polytetrafluoroethylene (ePTFE) or nickel and titanium alloy strands that may be arranged in a cylindrical, helically wound biaxial braid. The enlarged free-state diameter of the stent may be provided by the rest-state diameter of the metal helix or helices which support the braided polymer strands of the tubular mesh. The delivery device may retain and/or restrain the endoprosthesis with its ends longitudinally displaced from the free state resulting in a compact diameter residing snugly along the outer surface of the delivery device. When released from this elongated, small-diameter state, the diameter may be gained by increasing the angle between the warp and weft threads at their crossing points, in exchange for shortening the distance between opposite ends of the tube.

To insert an endoprosthesis, as herein described, using the herein disclosed delivery catheter assembly 1, a guide wire 3 may first be inserted, through the delivery catheter assembly 1, into a bodily vessel, such that a delivery catheter 4, preferably having at least one elongated tubular channel, may be guided along the guide wire 3, whereby the delivery catheter 4 will reach the bodily site in need of a stent. In a preferred embodiment, the delivery catheter 4 may be detachably secured to an endoprosthesis 20, such that the catheter 4 may deposit the endoprosthesis 20 at the site of a blockage. In some embodiments, once catheter 4 and detachably affixed endoprosthesis 20 reach the site of the blockage, a radio-opaque dye may be injected, via the delivery catheter assembly 1. In such embodiments, the radio-opaque dye may be applied at the site of the blockage, via channel 31 set within the delivery catheter, such that the endoprosthesis, guide wire, and catheter may be repositioned until the endoprosthesis is appropriately positioned.

The means for detaching the endoprosthesis from the delivery catheter may be actuated, via the delivery catheter assembly 1, to free the endoprosthesis, which may radially expand to contact and support the vascular walls and thus maintain vascular patency. In some embodiments, the endoprosthesis may remain permanently set within the vascular walls, maintaining vascular patency over the life of the patient. When deployed in blood vessels, such a prosthetic prosthesis may be called a self-expanding vascular stent.

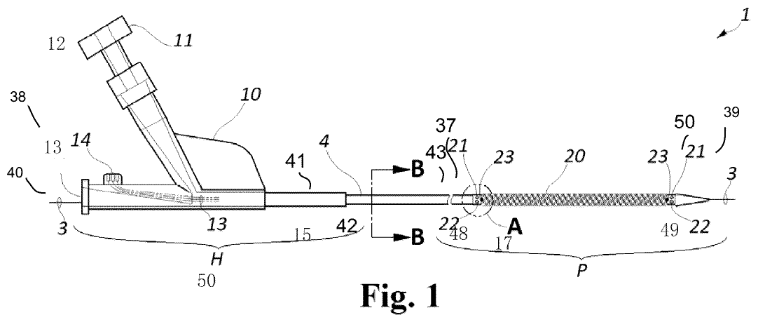

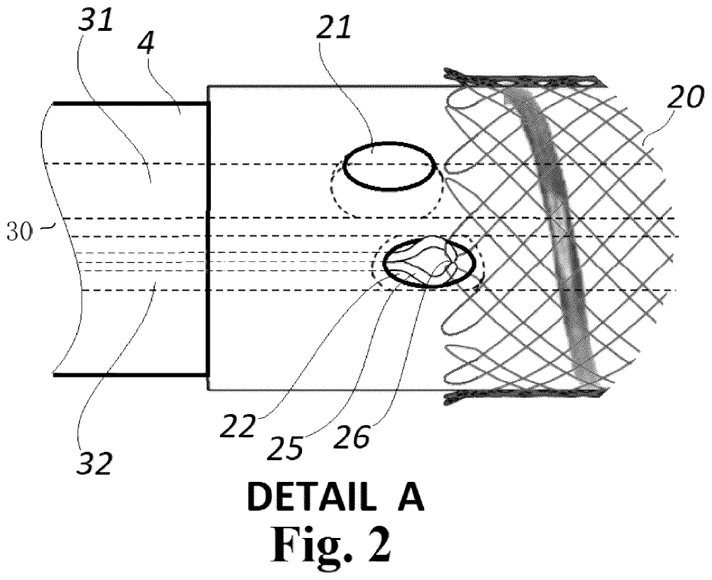

Referring to the figures, FIG. 1 illustrates a delivery catheter assembly 1 according to an embodiment of the disclosure. The delivery catheter assembly 1 may also be referred to as endovascular endoprosthesis delivery device 1 or stent delivery device 1. Endovascular endoprosthesis delivery device 1 may include a handle section 10, preferably set at a proximal end 38, and a delivery catheter 4, which may also be known as stent delivery sheath 4, such that catheter 4 may be connected to the handle section 10 and may extend to distal end 39 of the endovascular endoprosthesis delivery device 1. A detailed portion “A” of the endovascular endoprosthesis delivery device 1, showing a zoomed-in section of catheter 4 is shown enlarged in FIG. 2.

A plurality of channels 30, 31, and 32 may traverse from handle section 10 of endovascular endoprosthesis delivery device 1 through the length of delivery catheter 4, extending into distal end 39. In some embodiments, channel 30 may serve as a channel for guide wire 3, such that guide wire 3 passes through the entirety of channel 30. In some embodiments, channel 31 may function to funnel a radio-opaque dye through delivery catheter 4 to the site of a blockage. In some embodiments channel 32 may function as a means for detaching endoprosthesis 20 from endoprosthesis delivery device 1. Views of the aforementioned channels 30, 31, and 32 are illustrated in cross-section views 3a, 3b, and 3c.

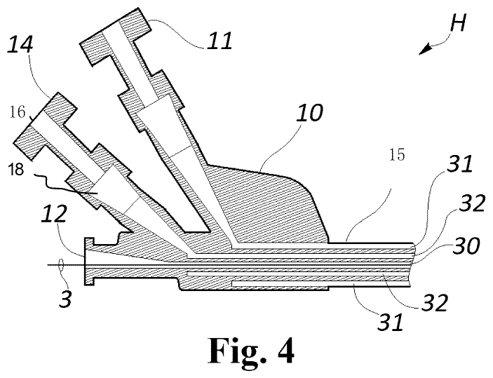

FIG. 4 shows a cross-section view of an alternative embodiment of a handle portion 10 of an endovascular endoprosthesis delivery device 1 in accordance with the invention, which may have three ports. The elongated body section 15 extending from the handle 10 may include a plurality of elongated tubular channels 30, 31, and 32 in its interior. Although the cross-section may be shown as a uniform pattern throughout this view, it may be understood that the handle section 10 may be a conglomeration of subcomponents that are assembled by solvent bonding, ultrasonic welding, or by other means suitable for medical product manufacturing which allow for efficacious sterilizing mediums and processes.

### Endovascular Endoprosthesis Delivery Device

As seen in FIG. 1, endovascular endoprosthesis delivery device 1 may include a first elongated tubular channel 30, such that guide wire 3 may pass through first elongated tubular channel 30, passing into and through delivery catheter 4. In some embodiments, first elongated tubular channel 30 may have a first end 40, such that first end 40 is set proximal to handle section 10 of endovascular endoprosthesis delivery device 1. First elongated tubular channel 30 may have a second end 41, such that second end 41 may be set distal to handle section 10 of endovascular endoprosthesis delivery device 1.

As seen in FIG. 1, in some embodiments, delivery catheter 4 may have a proximal end 42, set adjacent to first elongated tubular channel second end 41. Delivery catheter proximal end 42, may be coupled to and set adjacent to handle section 10. Additionally, catheter 4 may have catheter distal end 37. In a preferred embodiment, delivery catheter tip 43 may be set at delivery catheter distal end 37.

### Handle Section

In some embodiments, as seen in FIG. 1, handle section 10 may be a branched body 50. Branched body 50 may have a first terminus 11, such that first terminus 11 may function as a port for injecting contrast dye, including but not limited to a radio-opaque contrast dye into catheter 4. In some embodiments, first terminus 11 may include plunger 12, such that plunger 12 may be compressed to inject a radio-opaque fluid through delivery catheter 4, thereby delivering a contrast dye to a bodily site in need of endoprosthesis 20. In such embodiments, a dye may be inserted into contrast injection port 14 before being injected by plunger 20. Handle section 10 may include second terminus 13 such that second terminus 13 functions as a port for receiving guide wire 3. In some embodiments, second terminus 13 may house first elongated tubular channel 30, such that guide wire 3 may be inserted into second terminus 13 whereby guide wire 3 enters first elongated channel 30. Guide wire 3 may traverse the length of elongated channel 30, thereby traversing the length of delivery catheter assembly 1 from delivery catheter assembly proximal end 38 to delivery catheter assembly distal end 39. In some embodiments, first elongated tubular channel 30 may be set within second terminus 13 from second terminus first end 40, extending into delivery catheter tip 43. In some embodiments, first elongated tubular channel 30 may traverse the length of the delivery catheter 4.

In some alternative embodiments, guide wire 3 may also be incorporated into its own assembly which may include a seal or stopper member complementary to second terminus 13, such that the seal or stopper may prevent body fluids, and other fluids from entering second terminus 13. Such configurations may also be known as an over-the-wire (OTW) delivery system. In a preferred embodiment, delivery catheter assembly 1 may be detachably affixed to the endoprosthesis or stent 20. In such embodiments, endoprosthesis 20, may be attached to delivery catheter tip 43, and to delivery catheter assembly 1 at distal attachment site 50. It is preferable that a means for deploying endoprosthesis 20 be set within channel 32, such that endoprosthesis 20 may be detached from delivery catheter assembly 1 once endoprosthesis 20 is properly positioned within the body.

While the first elongated tubular channel 30 may function as a channel for guide wire 3 to pass through, and third channel 32 may function as housing for an endoprosthesis deployment means, second elongated tubular channel 31 may function as a means for funneling radio-opaque dye from first terminus 11 to the site of a clot or other such site in need of stent 20. In some embodiments, as seen in FIG. 2, second elongated tubular channel 31 of delivery catheter 4 may include a plurality of contrast delivery pores 21 that may be located near endoprosthesis proximal end 48 (as seen in FIG. 1) and endoprosthesis distal end 49 (as seen in FIG. 2). Thereby, when the dye is injected into tubular channel 31, only a small amount of dye emerges from contrast delivery pores 21, at both ends of endoprosthesis 20, preventing an excess of dye from entering a patient's body and quickening the time it takes for dye to reach the site in need of an endoprosthesis. Thus, when compared to traditional stent insertion methods, less dye and less waiting time may be required for effectively visualizing the locations of the two ends 48 and 49 of the endoprosthesis 20 within a bodily vessel.

In an alternative embodiment, radio-opaque markers may be incorporated onto the endovascular endoprosthesis delivery device 1, preferably near endoprosthesis proximal end 48 and endoprosthesis distal end 49, such that endoprosthesis 20 may be visualized via radiography without injecting patients with to radiographic dyes. In such embodiments, side effects of the radio-opaque dye are reduced, and the wait time between dye insertion and dye arrival at the clot site is eliminated.

In some embodiments, the radio-opaque markers may be directly set upon endoprosthesis 20, preferably at endoprosthesis proximal end 48 and endoprosthesis distal end 49, such that even after endoprosthesis delivery device 1 is retracted, endoprosthesis 20 may be tracked and monitored via radiographic imaging. As seen in FIG. 1 and according to one embodiment of the invention, the delivery catheter 4 may include an elongated body section 15 connected to the handle section 10 distal to the first terminus 11. The first elongated tubular channel 30, second elongated tubular channel 31, and third elongated tubular channel 32 may continue from the handle section 10 through elongated body section 15. The delivery catheter 4 may also include an endoprosthesis delivery section 17 connected to the elongated body section 15 distal to the handle section 10. First elongated tubular channel 30, second elongated tubular channel 31, and third elongated tubular channel 32 may continue from the elongated body section into the endoprosthesis delivery section 17. A radio-opaque contrast delivery pore 21 may be positioned on an exterior surface of the elongated body section 15 and located proximally to the endoprosthesis delivery section 17. The endoprosthesis delivery section 17 may be configured to incorporate an endoprosthesis device 20.

In some embodiments, as seen in FIG. 4, the handle section 10 may preferably include a third terminus 16, such that third terminus 16 may be used as a port for controlling a deployment means, such that a deployment means may be actuated to detach the endoprosthesis 20 from the delivery device 1. In an alternative embodiment, a port for controlling a deployment means may be set along the first terminus 11 or second terminus 13. In some embodiments, for electrically controlled detachment, electrically conductive wires may be incorporated into the handle section 10. In some embodiments, electrically conductive wires may be preferably inserted at the third terminus 16. In such embodiments, an endoprosthesis deployment means may lead to the endoprosthesis 20 or to an electrically controlled release means at or near the distal end 39 of the delivery device 1.

In some embodiments, whether electrically or mechanically deployed, the endovascular endoprosthesis delivery device 1 may preferably further incorporate other apparatuses distal to the handle section 10, providing operator control for the deployment of the endoprosthesis 20. These devices may be tension-generating devices that may cause retention strands to part and release an endoprosthesis 20 attached to the delivery device 1 or may cause mechanically closed or pinched stent retention features to relax and open so as to release the portion of the endoprosthesis 20 which they control.

In specific embodiments directed to applications of an endovascular endoprosthesis delivery device 1 such as a vascular catheter system, as depicted in FIG. 1, the invention may be described as including a handle section 10 at the proximal end 38 of the vascular catheter system, an endoprosthesis delivery section 17 at the distal end 39 of the vascular catheter system, and an elongated body section 15 between the handle section 10 and the endoprosthesis delivery section 17. Within the endoprosthesis delivery section, there may be one or more radio-opaque contrast delivery pores 21. The elongated body section 15 may preferably include a plurality of elongated tubular channels, in which a first elongated tubular channel 30 may receive a guide wire 3 and a second elongated tubular channel 31 may act as a conduit to deliver radio-opaque contrast from the handle section 10 to the radio-opaque contrast delivery pores 21. “Contrast” in this specification defines any of the radio-opaque diagnostic drugs used in radiography for the enhancement of radiographic (x-ray) examinations. Although the word is commonly followed by “media” or “agent” as a technical term, in this specification the word “contrast” is used as a stand-alone noun encompassing these media and agents.

FIG. 2 shows an enlarged detail view of region “A” of the delivery catheter assembly 1 as defined in FIG. 1. The delivery catheter 4 may include a first elongated tubular channel 30 that may receive a guide wire 3 and a second elongated tubular channel 31 that may deliver radio-opaque dye proximal to the proximal end 48 and distal end 49 of the endoprosthesis 20 (as seen in FIG. 1), which may be detachably affixed to the delivery catheter 4. The second elongated tubular channel 31 may be connected to radio-opaque contrast delivery pores 21 located proximal to the endoprosthesis 20. In some embodiments, the radio-opaque contrast delivery pores 21 may be located on the exterior surface of the delivery catheter 4. Contrast delivery pores 21 may function to deliver radio-opaque contrast media substantially proximate to an in vivo target portion of the delivery catheter assembly 1, such that the location of the endoprosthesis device 20 may be better visualized during insertion or during a surgical procedure.

In some embodiments, a third elongated tubular channel 32 may incorporate endoprosthesis deployment means (not shown) which may be controllable and actuatable from outside the patient. Stent implantation is nowadays a common interventional procedure with a high rate of success if compared to angioplasty. Placing a stent may be done through a minimally invasive procedure known as a percutaneous coronary intervention (PCI) or angioplasty. A long, thin, flexible tube called a catheter may be inserted into blood vessels, often providing access to the heart. This tube may include an empty balloon at the end of it, which may be inflated with air to open the narrowed artery and place the stent. The stent may expand and lock in place to hold the artery open. A vascular stent is often a small slotted metal tube, mesh, or as otherwise known in the art. A stent may be inserted into an artery at the site of narrowing to act as an internal scaffolding or support to the blood vessel. A number of strategies for stent optimization have been proposed, including stent and post-stent balloon dilation sized to the reference segment's lumen dimensions; post-stent dilation with a balloon sized to the distal reference lumen diameter with expansion of proximal and mid stent by dilation with a balloon. A stent may be deployed in many of the standard means as are known in the art, and may include an elongated member sent with a shaft through or along a catheter, or otherwise sent through an orifice and/or vessel to the position for locating the and releasing or deploying a stent. In some embodiments, as seen in FIG. 2, a mechanical stent retention means 25 may be a looped strand of material emerging from a control pore 22, such that the looped strand of material is also coupled to third elongated tubular channel 32. The stent retention means 25 may include a fallible point 26 of structural weakness which may be a region having less tensile strength than the rest of the material or, in some embodiments, a region of sharply reduced cross-sectional area which may act as a stress concentrator. For an embodiment using mechanical controls to deploy an endoprosthesis 20, the third tubular channel lumen 32 may connect to a third terminus 16 of the handle section 10 (as seen in FIG. 4) of the endovascular endoprosthesis delivery device 1, such that third terminus 16 may be set with controls for deploying endoprosthesis 20. In such embodiments, third terminus 16 may incorporate a medical mechanical interconnect such as a Luer connection for allowing access for tensile control of the stent retention means 25, such that endoprosthesis 20 may be retained on the delivery catheter 4 until it is in position for deployment. In such embodiments, to deploy endoprosthesis 20, tension or a tensile impulse may be applied to the stent retention means 25, via third terminus 16, such that an end of endoprosthesis 20 may be freed from catheter 4.

Alternatively, endoprosthesis 20 may be detached from catheter 4 via electronic controls. In such embodiments, a current-carrying material having an abrupt change in resistivity at a predetermined location, fallible point 26, may be set within third channel 32. In such embodiments, when current is applied to the current-carrying material, an intense localized i2R heating may melt the strand material at the fallible point 26, causing the material to break, or otherwise separate, such that endoprosthesis 20 may be deployed from the delivery catheter 4. Whether mechanically or electrically deployed, or otherwise, the controls may be configured to deploy a self-expanding vascular endoprosthesis 20 upon operation of the controls, as is known in the art.

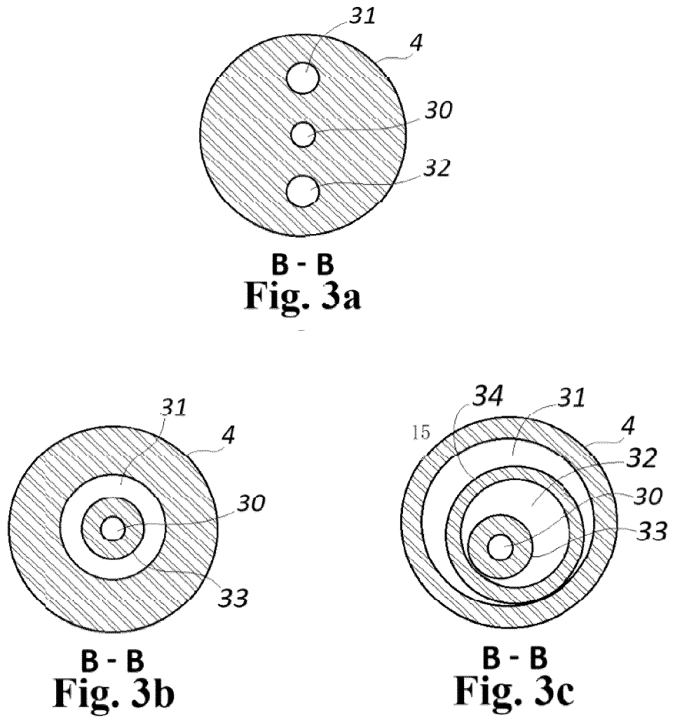

FIG. 3a shows a cross-section view of the delivery catheter assembly 1 of FIG. 1 taken at section line “B-B” defined in FIG. 1. The delivery catheter 4 may include a plurality of elongated tubular channels or lumina in the interior of the elongated body section 15, including a first lumen 30 for receiving and following a guide wire 3 and a second lumen 31 for delivering radio-opaque contrast to radio-opaque contrast delivery pores 21 which may be set proximal to the ends 48 and 49 of the endoprosthesis 20. In preferred embodiments, endoprosthesis 20 may be set near distal end 43 of delivery catheter 4. A third lumen 32 may function to contain a deployment means 18 (as is known in the art), such that a deployment means may be actuated for detaching and deploying the endoprosthesis 20. Alternative embodiments may include an additional lumen for deployment means 18 such that the endoprosthesis proximal end 48 and the endoprosthesis distal end 49 may each have their own independent or dedicated detachment control, residing in their own channels. Although a preferred embodiment may have a first elongated tubular channel 30 for receiving and following a guide wire 3 in the center of the circular cross-section of the delivery catheter 4 and the other elongated tubular channels located axially symmetrical to the center elongated tubular channel, it is also within the scope of the invention to position the elongated tubular channels in other locations within the cross-section, including, but not limited to, within a centrally defined triangle or other geometrical region and having no elongated tubular channels passing through the center of the cross-section. In an exemplary embodiment, a first elongated tubular channel 30 may be configured to receive a guide wire 3 through the length of the first elongated tubular channel 30, and a second elongated tubular channel 31 may connect to the first terminus 11, such that first terminus 11 may function as a radio-opaque contrast injection port and a radio-opaque contrast delivery pore 21.

In such alternative embodiments, the elongated body section 15 may include a plurality of elongated tubular channels, including but not limited to a first elongated tubular channel 30 that may extend from second terminus 13 to distal end 43 of delivery catheter 4, such that a guide wire may pass through first tubular channel, and continue through endoprosthesis 20. Elongated body section 15 may also have a second elongated tubular channel 31, such that second elongated tubular channel 31 may begin at first terminus 11, such that first terminus 11 may function as a radio-opaque contrast injection port, extending from handle section 10 to radio-opaque contrast delivery pore 21. In some embodiments, contrast delivery pore 21 may be set on an exterior surface of delivery catheter 4. Elongated body section 15 may also include a third elongated tubular channel 32, such that third elongated tubular channel 32 may function to contain a mechanism for controlling the deployment of endoprosthesis device 20. The third elongated tubular channel 32 may begin at a third terminus 16, as seen in FIG. 4, such that third terminus 16 may function as a port to operate, actuate, or control the endoprosthesis deployment means. It is preferable that the endoprosthesis deployment means function from handle section 10 through control pore 22, such that the deployment means can actuate the separation of endoprosthesis 22 from endoprosthesis delivery device 1 at control pore 22.

In an alternative embodiment, as seen in FIG. 3b, a first interior tube 33 may be arranged concentrically within the lumen of delivery catheter 4, thereby defining a first elongated tubular channel 31 within the first interior tube 33 and a second elongated tubular channel 30 which contains the first interior tube 33. In such alternative embodiments, each elongated tubular channel 30, 31, and 33 may be arranged concentrically within delivery catheter 4. It may be understood that the first interior tube 33 may be free to migrate within the second elongated tubular channel 30 of the delivery catheter 4, such that the meaning of the word “concentric” in the context of the present specification may be expanded beyond its literal meaning of the centroids of certain nested objects being exactly concentric or coaxial. Rather, in this specification the word may also encompass broader meanings of objects recursively nested within each other, or successively circumscribed one by the next, such as the nested sections of a set of tubular objects. Other similar examples beyond the scope of the invention include concentric sections of a telescoping radio antenna or a collapsible baton.

As seen in FIG. 3b elongated body section 15 between handle section 10 and the radio-opaque contrast delivery pores 22 of delivery catheter assembly 1 of FIG. 1 may include a plurality of channels, in which a first elongated tubular channel 30 may be used for receiving a guide wire 3 and may be circumscribed by a second elongated tubular channel 31, which may deliver radio-opaque contrast from the handle section 10 to the radio-opaque contrast delivery pores 22.

FIG. 3c shows a cross-section view of another alternate embodiment of a delivery catheter assembly 1 taken at section line “B-B” defined in FIG. 1 which may include three concentrically nested tubes such that the first interior tube 33 may be nested within a second interior tube 34, such that e both interior tubes 33 and 34 may be nested within the delivery catheter 4, thereby defining a first elongated tubular channel 30, a second elongated tubular channel 31, and a third elongated tubular channel 32 all arranged concentrically within the elongated body section 15. The ordinal sequence of these structures may be arbitrary and may be re-ordered such that the outermost channel is designated as the first elongated tubular channel, with the second elongated tubular channel within the first, and the third elongated tubular channel within the second channel of the elongated body section of having the plurality of tubular channels.

FIG. 4 shows a cross-section view of an embodiment of a three-sectioned handle portion 10 of an endovascular endoprosthesis delivery device 1. Elongated body section 15 may extend from handle 10, such that elongated body section 15 may include a plurality of elongated tubular channels in its interior. A first elongated tubular channel 30 may receive a guide wire 3 throughout its length. Guide wire 3 may be inserted into the endoprosthesis delivery device via second terminus 13. In some embodiments, second terminus 13 may function as a flanged guide wire port. A second elongated tubular channel 31 may connect first terminus 11, such that first terminus 11 may function as a radio-opaque contrast injection port coupled to a radio-opaque contrast delivery pore 21 such as pore 21 of FIG. 2. A third elongated tubular channel 32 may contain a mechanism for controlling deployment of an endoprosthesis 20, wherein the mechanism for controlling deployment of the endoprosthesis device 20 may be a stent retention means 25 that may preferably be a loop portion of a strand which may include a fallible point 26, such that fallible point 26 may be activated by mechanical tension or electrical current. Access at the handle section 10 to this third elongated tubular channel 32 may be provided at third terminus 16 which may be used as a port for the control of endoprosthesis deployment means. Although the cross-section hatching is shown as a uniform pattern throughout this view, it may be understood that the handle section 10 may be a conglomeration of subcomponents that are assembled by solvent bonding, ultrasonic welding, or by other means suitable for medical product manufacturing which allow for efficacious sterilizing mediums and processes.

Methods of use of the invention may follow from the foregoing descriptions of their various structural embodiments. For example, a method of inserting an endoprosthesis 20 into a blood vessel, may include the step of providing an endovascular endoprosthesis delivery device 1, which may include a handle section 10 at a proximal end 38 of the endovascular endoprosthesis delivery device 1. The handle section 10 may also include a first terminus, which may be used as a guide wire port for receiving a guide wire into the interior of the endovascular endoprosthesis delivery device 1, of the handle section 10 and a second terminus 13, which may be used as a radio-opaque contrast injection port to permit the injection of a radio-opaque contrast fluid into the interior of the endovascular endoprosthesis delivery device 1. The endovascular endoprosthesis delivery device 1 may also include an endoprosthesis delivery section 17 at the distal end 39 of the endovascular endoprosthesis delivery device 1 and an elongated body section 15 between the handle section 10 and the endoprosthesis delivery section 17. The exemplary method may also include the steps of inserting a guide wire 3 into arterial access, fitting the tip 43 of the endovascular endoprosthesis delivery device 1 over the guide wire 3 into the arterial access until the endoprosthesis delivery section 17 reaches a deployment target of the endoprosthesis 20, wherein a radio-opaque contrast delivery pore 21 is positioned upstream of the target, injecting radio-opaque contrast fluid into a first terminus 11, which may be a radio-opaque contrast injection port, and delivering radio-opaque contrast to a radio-opaque contrast delivery pore 21, permitting the radio-opaque contrast to flow over the endoprosthesis 20 at the deployment target. An optional extension of the method may include the steps of deploying the endoprosthesis 20 at the deployment target by actuating deployment means located on the handle section 10 and removing the endovascular endoprosthesis delivery device 1 and the guide wire 3 from the arterial access.

Another optional variation of the method within the scope of the invention may be feasible wherein the endoprosthesis 20 may contain a radio-opaque marker on a distal end 49 of the endoprosthesis 20 and a radio-opaque marker on the proximal end 48 of the endoprosthesis 20, and wherein the step of deploying the endoprosthesis 20 at the deployment target by actuating a deployment means located on the handle section 10 may include confirming the location of the endoprosthesis 20 at the target through application of an imaging device to view the radio-opaque contrast and radio-opaque markers, and actuating the deployment means 18 to permit the expansion of the endoprosthesis 20 at the deployment target.

Similarly, the inventive method may be practiced using an alternative apparatus previously described wherein the handle section 10 may further include a third terminus 16 of the handle section 10 of the endovascular endoprosthesis delivery device 1 which may provide a secure and efficacious connection for controls which affect the deployment means of the endoprosthesis 20, such as by material rupture or mechanical release of a stent retention means 25, or by application of an electrical current through an electrical conductor which may include a first cross-section throughout most of its length and a reduced cross section or a material treatment effecting an elevated bulk resistance at a second, fusible portion of its length, so that local resistance heating may cause the stent retention means 25 to part.

Inspection and combination of the novel features disclosed in FIG. 1 and FIG. 3b may reveal another optional method of practice wherein the elongated body section 15 of the endovascular endoprosthesis delivery device 1 provided above may further include a plurality of elongated tubular channels, wherein a first elongated tubular channel 30 may receive a guide wire 3 through the length of the first elongated tubular channel 30 and a second elongated tubular channel 31 may connect the first terminus 11 and a radio-opaque contrast delivery pore 21, and wherein the first elongated tubular channel 30 and the second elongated tubular channel 31 are arranged concentrically within the elongated body section 15, notwithstanding bending effects whereby with nested structures such as those of FIGS. 3b and 3c, if the elongated body section 15 is bent into an arc while navigating within a bodily cavity, the nominally concentric tubes may migrate out of geometrically concentric alignment while remaining in a “nested” structure encompassed by the definition of “concentric” in this specification.

While certain features and aspects have been described with respect to exemplary embodiments, one skilled in the art will recognize that numerous modifications are possible. Also, while certain functionality is ascribed to certain system components unless the context dictates otherwise, this functionality may be distributed among various other system components in accordance with the several embodiments.

Moreover, while the procedures of the methods and processes described herein are described in a particular order for ease of description unless the context dictates otherwise, various procedures may be reordered, added, and/or omitted in accordance with various embodiments. Furthermore, the procedures described with respect to one method or process may be incorporated within other described methods or processes; likewise, system components described according to a particular structural configuration and/or with respect to one system may be organized in alternative structural configurations and/or incorporated within other described systems.

The present disclosure is not to be limited in terms of the particular embodiments described in this application, which are intended as illustrations of various aspects. Many modifications and variations may be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled.

Hence, while various embodiments are described with or without certain features for ease of description and to illustrate exemplary aspects of those embodiments, the various components and/or features described herein with respect to a particular embodiment may be substituted, added, and/or subtracted from among other described embodiments, unless the context dictates otherwise. Thus, unauthorized instances of apparatuses and methods claimed herein are to be considered infringing, no matter where in the world they are advertised, sold, offered for sale, used, possessed, or performed.

Consequently, and in summary, although many exemplary embodiments are described above, it will be appreciated that the invention is intended to cover all modifications and equivalents within the scope of the following claims.

I claim:

1. A stent delivery device comprising;
a handle section set at a proximal end of the stent delivery device;
an elongated body section coupled to said handle section, said elongated body section set distally of said handle section and coupled with a catheter;
a stent coupled to said catheter, such that said stent is set between said catheter and a distal end of said stent delivery device;
a first channel set within said stent delivery device, said first channel comprising a guide wire passing therethrough;
a second channel set within said stent delivery device, said second channel comprising a contrast liquid passing through; and
a third channel set within said stent delivery device, said third channel comprising a deployment means passing through.

2. The stent delivery device of claim 1, wherein the handle section comprises a dual-branched body.

3. The stent delivery device of claim 2, wherein the handle section comprises a first branch having a first terminus, wherein said first terminus leads into the first channel, whereby the guide wire may be inserted into said first terminus such that said guide wire may pass through said first terminus into said first channel.

4. The stent delivery device of claim 2, wherein the handle section comprises a second branch comprising a second terminus, wherein said second terminus comprises a contrast dye injection port, whereby said contrast dye injection port is configured allow injection of a contrast dye through said injection port into said second channel set within said stent delivery device, whereby said contrast dye may pass through the contrast dye injection port through said second channel and into said stent.

5. The stent delivery device of claim 2, wherein said handle section comprises a third branch having a third terminus, such that said third terminus comprises an endoprosthesis deployment port comprising controls for the mechanical and/or electric deployment of the stent.

6. The stent delivery device of claim 5, wherein said third terminus leads to the third channel, such that a mechanism for releasing the stent from the stent delivery device passes into said third terminus, through said third channel, into a deployment target set adjacent to or within said stent near the distal end.

7. The stent delivery device of claim 1, wherein said elongated body section is configured with a plurality of channels passing through said elongated body section, such that the plurality of channels pass from said handle section, into said elongated body section, and out of said elongated body section into said catheter.

8. The stent delivery device of claim 7, wherein the plurality of channels comprises at least the first channel configured to receive a guide wire, the second channel configured to receive contrast dye, and the third channel configured to receive a stent deployment means.

9. The stent delivery device of claim 8, wherein the first channel, the second channel, and the third channel are arranged concentrically within the elongated body section.

10. The stent delivery device of claim 9, wherein the second elongated tubular channel is the outermost channel within the elongated body section of the plurality of tubular channels.

11. The stent delivery device of claim 8, wherein the first channel, the second channel, and the third channel are arranged in parallel within the elongated body section.

12. The stent delivery device of claim 1, wherein the second channel passes from said handle section, into said elongated body section, and through into said catheter into said stent, whereby said stent may be inserted into a patient via said guide wire, whereby said catheter may deliver a contrast dye to said stent such that the contrast dye reaches an area in need of said stent.

13. The stent delivery device of claim 1, wherein said catheter may deliver a contrast dye to an area in need of contrast dye via at least one pore set within a side of said stent, whereby said contrast dye flows from said catheter into said stent, exiting said stent via the at least one pore, and entering the area in need of a contrast dye via the at least one pore set within said stent.

14. The stent delivery device of claim 1, wherein said stent may be set with radio-opaque markers.

15. A method of inserting a stent into a bodily vessel, the method comprising:
inserting a guide wire set within a stent delivery apparatus into a bodily vessel;
guiding the stent coupled to the stent delivery apparatus via the guide wire until the stent reaches a target area;
injecting a contrast dye through the stent delivery apparatus whereby the contrast dye travels from a handle in the stent delivery apparatus through a channel in the stent delivery apparatus into the stent, whereby the contrast dye exits the stent into the target area via at least one pore in said stent;
viewing the target area via radiographic imaging and adjusting the stent location via the guide wire until the stent is properly positioned;
disengaging the stent from the stent delivery apparatus and removing the guide wire and the stent delivery apparatus from the target area.
